(19) United States
(12) Patent Application Publication
(10) Pub. No.: US 2017/0319806 A1
(43) Pub. Date: Nov. 9, 2017

(54) Mouth Leak Prevention Device and Leak Prevention Method

(71) Applicants: Harold J. Teller, Lebanon, CT (US); Nicholas Serignese, Lebanon, CT (US)

(72) Inventors: Harold J. Teller, Lebanon, CT (US); Nicholas Serignese, Lebanon, CT (US)

(21) Appl. No.: 15/272,814

(22) Filed: Sep. 22, 2016

Related U.S. Application Data
(60) Provisional application No. 62/331,510, filed on May 4, 2016.

(51) Int. Cl. A61M 16/06; A61M 16/08; A61M 16/00

(52) U.S. Cl. CPC A61M 16/06; A61M 16/0057; A61M 16/0816

(57) ABSTRACT

A new innovative and unique solution to minimize the problems of mouth leakage using a CPAP machine is disclosed. A mouthpiece module is specifically designed to solve the problem of mouth leakage which compromises the therapeutic effect of CPAP treatment. The mouthpiece module comprises a flexible absorbent material which partially wraps around the lips and is secured to the back of the head by straps or an elastic band. In one embodiment, the mouthpiece is detachable from a securement strap.

### BACKGROUND

Continuous Positive Airway Pressure (CPAP) is an effective treatment for sleep apnea. A CPAP machine delivers a positive pressure on airways of a patient. The positive pressure keeps the patient's airways open to avoid cessation of breathing related to airway closure that may occur in various stages during the sleep of a patient.

The present disclosure relates to a mouthpiece that is specifically designed to be used for the prevention of mouth leaks that can occur with the use of a CPAP machine that is used as a treatment in therapy for sleep apnea.

The airflow pressure produced by a CPAP machine is higher when compared to normal breath pressure and, as a result, a major problem that can be experienced by the patient is air leakage from the mouth. Mouth leaks, if not corrected, can compromise the patient's treatment to the extent that the treatment can become ineffective. Other effects can be that the patient complains of a dry mouth which is caused by the CPAP air escaping through the mouth. This is brought about by the effect of higher than normal breathing pressure which tends to “dry out” the oral and nasal cavity due to the CPAP pressure being exhaled through the mouth rather than being delivered to the patient's lungs via the airway and exhausted through the exhaust vent of the CPAP mask. The result of this is that the CPAP pressure can be compromised and adequate pressures are not maintained to optimize the patient's treatment.

At present, there are four well-known options that can be used by patients to minimize the problem of mouth leakage. The first of these methods is for a patient to learn to maintain a closed mouth without an aid. To this end, some people position the tongue behind the top teeth which forces the back of the tongue to spread towards the side of the mouth creating a seal so that the CPAP works. The second option is to use a full face mask. If a person typically breathes through the mouth, a full face mask is almost always required. A third option is the use of a chin strap. Chin straps are a simple device and comprise a chin support pouch made from fabric with straps fitted that are secured in position over the top of the head. The disadvantage of a chin strap is that it can be quite uncomfortable to wear, leaves strap marks on the face after prolonged use, can become loose during sleep with patient movement and can still allow mouth leakage if not adjusted and tightened correctly. The fourth method is mouth taping. Mouth taping is typically a desperation method and very unadvisable. Aside from being inconvenient and uncomfortable, mouth taping can cause a very unsafe condition if a patient's nose becomes obstructed, the patient has acid reflux, the patient regurgitates or the CPAP is disconnected and the patient needs to breathe through the mouth.

Continuous Positive Airway Pressure (CPAP) machine users as they grow older commonly experience a problem known as mouth leakage that develops with their lips losing a degree of elasticity. When using their CPAP with a nose mask, their lips, through a loss of elasticity, open from the air pressure produced by the CPAP machine as they approach REM sleep. The air forcibly pushes its way through their lips, causing them to separate slightly, thereby resulting in a hissing sound, trembling and fluttering lips. The characteristics can impact the effective functionality of the CPAP machine to provide the necessary air flow for adequate breathing for apnea patients.

### BRIEF SUMMARY OF THE INVENTION

Briefly stated, a mouth leak prevention device keeps the lips of a user sealed while using a nasal mask for a CPAP. The mouthpiece of this device has two opposite edges that generally traverse its length and two opposite ends that generally traverse its width. The mouthpiece is made of flexible and absorbent material. The mouthpiece is wider than the closed lips of a user. The device also has a pair of straps that extend from the ends of the mouthpiece a distance that is sufficient to ensure that the straps are secured at the back of the head of a user. In use, the straps are securable at the back of the head of the user, as the mouthpiece is extended in between and around portions of the lips of a user, to create an airtight seal at the mouth of the user.

In one embodiment, the mouth leak prevention device can be made of a cotton cloth material. The device can also be made of various cloth materials, polyester, hemp, cotton, various composite textile materials or any combination thereof. The straps of the mouthpiece extend out from the ends of the mouthpiece as one piece or multiple pieces of material made substantially of the same material as the mouthpiece.

In another embodiment, a mouth leak prevention device also keeps the lips of a user sealed while using a nasal mask for a CPAP and employs a mouthpiece. The mouthpiece has two opposite edges that generally traverse along its length and two opposite ends that generally traverse its width. The mouthpiece is made of flexible and absorbent material. The mouthpiece is wider than the closed lips of a user. The device also has a pair of straps that extend from the ends of the mouthpiece a distance that is sufficient to ensure that the straps are securable at the back of the head of a user. There is also a hook-and-loop fabric fastener on a portion of the straps to allow the mouthpiece to be removed and replaced. In use, the straps are secured at the back of the head of the user, as the mouthpiece is extended in between and around portions of the lips of a user, to create an airtight seal at the mouth of the user.

In this second embodiment, the mouth leak prevention device can be made of a cotton cloth material or various other materials. The straps of the mouthpiece extend out from the ends of the mouthpiece as one piece or multiple pieces of material made substantially of the same material as the mouthpiece. A hook-and-loop fabric fastener can be attached to a portion of one of the straps or both of the straps. The hook-and-loop fabric fastener can be positioned at the portion of one or both straps either next to the ends of the mouthpiece or at the ends of the straps next to where the straps are securable to the back of the head of the user.

In another embodiment, a mouth leak prevention device which keeps the lips of a user sealed while using a nasal mask for a CPAP employs a removable mouthpiece. The mouthpiece of this device has two opposite edges that traverse its length and two opposite ends that traverse its width. The mouthpiece is made of flexible and absorbent material. The mouthpiece is wider than the closed lips of a user. The device also has a securement means for securing the mouthpiece to the back of the head of a user. When in use, the mouthpiece is secured at the back of the head of the user, as the mouthpiece is extended in between and around portions of the lips of a user, to create an airtight seal at the mouth of the user. The securement means may also have features which allow for detachment of the mouthpiece.

### BRIEF DESCRIPTION OF DRAWINGS

FIG. 1 is a perspective view of a mouthpiece module for a CPAP machine;

FIG. 2 is a frontal head view, partly in schematic, of a user wearing a nasal mask and the mouthpiece with the cloth portion in the mouth and sections wrapped around the head and tied in the back of the head and schematically illustrating a CPAP machine;

FIG. 3 is an enlarged side view, partly in schematic, of the mouthpiece module installed in the mouth;

FIG. 4 is a photograph of one embodiment of a mouthpiece module;

FIG. 5 is an exploded diagrammatic view of another embodiment of a mouthpiece module; and

FIG. 6 is an exploded view of yet another embodiment of a mouthpiece module.

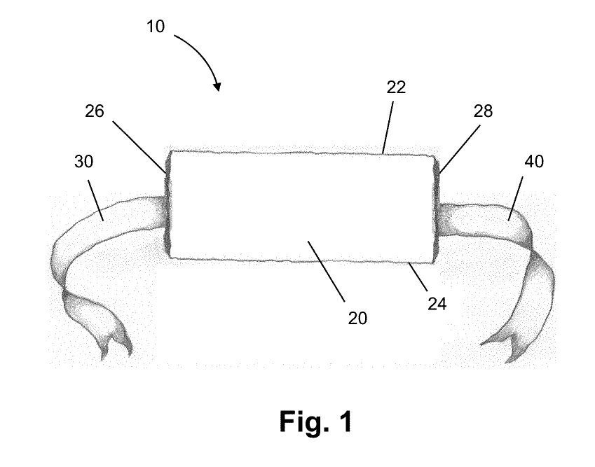

### DETAILED DESCRIPTION

With reference to the drawings wherein like numerals represent like parts throughout the figures, a mouthpiece module, in accordance with the present disclosure, is generally designated by the numeral 10. The mouthpiece module functions in conjunction with a nasal mask 12 for a CPAP machine 14 to prevent air leakage from the mouth while a user is using a CPAP machine. The nasal mask 12 can take many forms, and nasal mask 12 is intended to be representative of any of the various forms which can be employed.

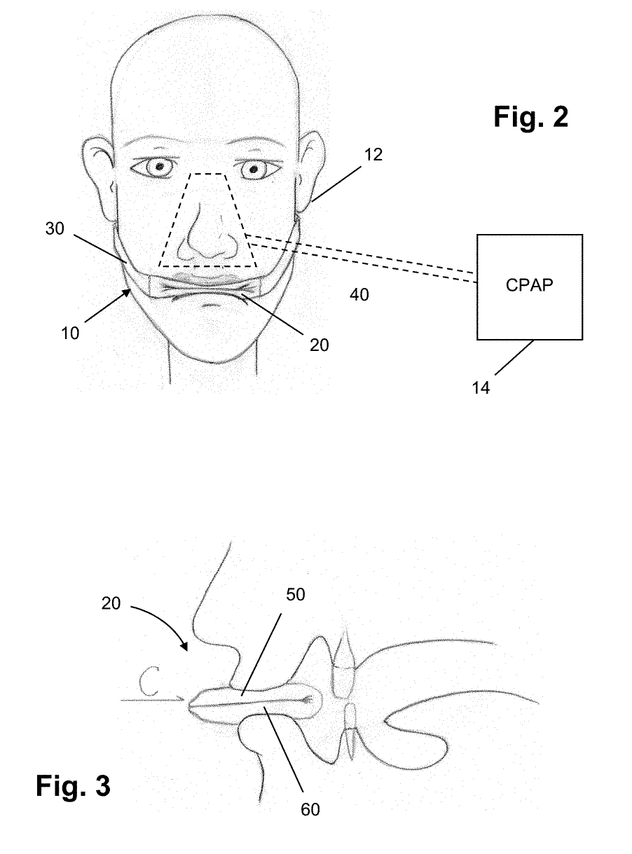

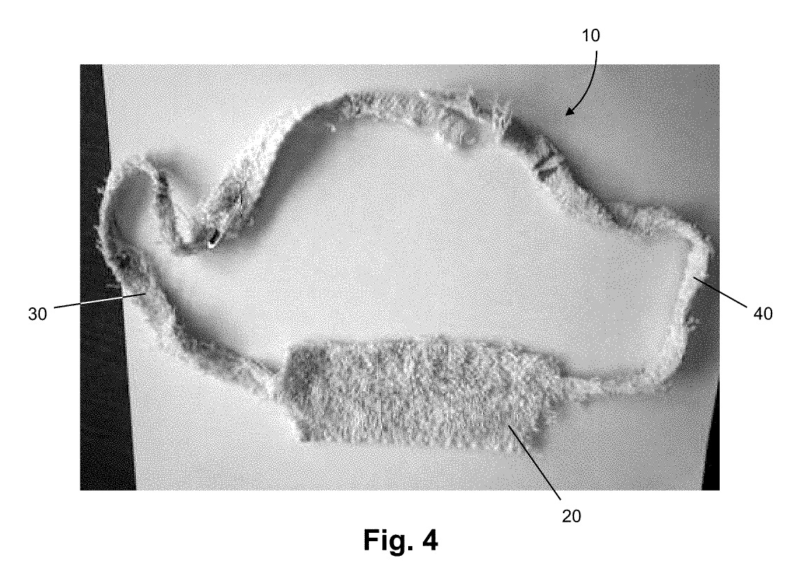

The mouthpiece module 10 is manufactured from a flexible absorbent cloth or textile material. The mouthpiece module is configured and dimensioned to rest between the lips of the user, as best represented in FIG. 2, and does not extend beyond the teeth. The preferred cloth or textile material for the mouthpiece module is preferably cotton, but may be polyester. Bamboo, fleece, fabric, hemp or elastic or other flexible absorbent materials may be employed. The preferred length of the mouthpiece module is 30-40 inches. Naturally, the mouthpiece module is healthful, clean and safe.

The mouthpiece module 10 comprises a rectangular, quasi-rectangular, oval or generally elongated mouthpiece 20. The mouthpiece has a top edge 22, an opposed second edge 24 and opposed ends 26 and 28. The mouthpiece has a length which exceeds the length of the lip interface of the user when the lips are closed. The preferred dimensions of the mouthpiece 20 are 1-3 inches in width and 3-5 inches in length.

A pair of straps 30 and 40 extend from a medial location of the opposed ends 26 and 28. In one embodiment, the straps 30 and 40 each have a length which is more than sufficient to extend around the head of the user and to provide sufficient end material for comfortably tying the straps in a knot at the back of the head of a user. The preferred width of the straps 30 and 40 is ¼-½ inch. The straps 30 and 40 are non-irritating and comfortable.

The ends of the straps may alternatively be secured by a pin, clasp or various fasteners or, in some embodiments, even by an elastic connector or hook-and-loop fabric (Velcro™) fastener. In addition, as best illustrated in FIG. 2, the upper and lower flexible panel portions 50 and 60 of the mouthpiece 20 engage around the upper and lower lips of the user in a reverse fold fashion, as best illustrated in FIG. 3.

In some embodiments, the straps may be stitched to the ends of the mouthpiece 20. In other embodiments, the straps may simply integrally extend from the mouthpiece. In another embodiment, the straps may also be a single continuous elastic band, stitched or integral to the mouthpiece 20. Any of the above securing means are suitable for securing the straps at the back of the head so that the mouthpiece is firmly secured in the mouth.

Once the mouthpiece 20 is properly positioned and retained in place to the user, it will be appreciated that the mouthpiece provides a flexible comfortable structure which prevents airflow from the mouth. In addition, the absorbent material from the mouthpiece will absorb any moisture in the oral cavity which impinges in the lip area.

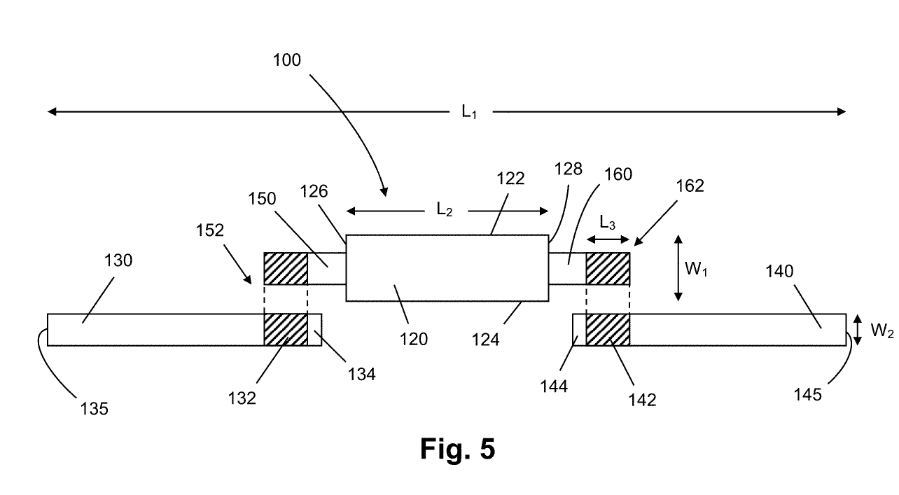

With reference to FIG. 5, another embodiment of a mouthpiece module is generally designated by the numeral 100. Mouthpiece module 100 comprises a rectangular or quasi-rectangular elongated mouthpiece 120. The mouthpiece module 100 is especially adapted so that the mouthpiece 120 may be dismounted and replaced by another or a new mouthpiece as required. In this regard, the mouthpiece may be replaceable or, in some cases, disposable.

The mouthpiece 120 has a top edge 122, an opposed second edge 124 and opposed ends 126 and 128. It should be appreciated that, depending on the mouthpiece geometry, the edges and ends may not have a well-defined point of demarcation. The mouthpiece 120 has a length L₂ (which exceeds the length of the lip interface of the user when the lips are closed) and has a width W₁. A pair of connectors or tab-like appendages 150 and 160 extend from a medial portion of the ends 126 and 128, respectively. The appendages may be integral with the mouthpiece, stitched or otherwise connected. The opposite ends of the appendages mount a fastener, such as a portion of a hook-and-loop or Velcro™-type fabric fastener 152 and 162, respectively. The length of each fastener is designated by the length L₃.

Straps 130 and 140 which, in one embodiment, are preferably separate segments, each include a mating fastener, such as a second Velcro™ fastener, hook-and-loop fabric, or Velcro™-type fastener portion 132 and 142 which connect with the fastener portions 152 and 162, respectively, in overlapping relationship. The straps 130 and 140 are preferably substantially identical and have a width designated as W₂. When the straps are connected to the appendages 150 and 160, the maximum longitudinal distance between the free ends of the straps is designated by L₁. The length L₁ is sufficient that the straps may be either tied or otherwise connected at the back of the head of the user. The straps may include various mechanical fasteners for fastening the straps at the back of the head of the user. The preferred maximum dimension L₁ is between 30 and 40 inches.

In one embodiment, the straps may essentially be connected end-to-end at ends 135 and 145 or seamlessly joined so that components 130 and 140 are essentially strap segments of continuous band and the strap has sufficient elasticity so that it can be wrapped around the back of the head of the user and be sufficiently taut to mount the mouthpiece module in a proper taut installed position, such as illustrated in FIG. 2.

Table 1 set forth below illustrates representative samples for various different dimensioned embodiments of mouthpiece module 100. The fasteners are connected to the straps 130 and 140 so that the proximal end of the straps include a tab 134 and 144 to allow for easy removal or disconnection of the straps from the tab appendages.

TABLE 1

| Ex. | W | W2 | L1 | L2 | L3 |
|---|---|---|---|---|---|
| 1 | 2 | ⅜ | 37 | 5 | ½ |
| 2 | 3 | ⅜ | 37 | 5 | ½ |
| 3 | 2 | ⅜ | 37 | 5 | 2 |
| 4 | 3 | ⅜ | 37 | 5 | 2 |
| 5 | 2 | ⅜ | 37 | 5 | 3 |
| 6 | 3 | ⅜ | 37 | 5 | 3 |
| 7 | 2 | ⅜ | 37 | 5 | 4 |
| 8 | 3 | ⅜ | 37 | 5 | 4 |

W1 - Width of mouthpiece (inches).
W2 - Width of straps (inches).
L1 - Length of entire mouth leak prevention device (inches).
L2 - Length of mouthpiece (inches).
L3 - Length of hook-and-loop fastener (inches).

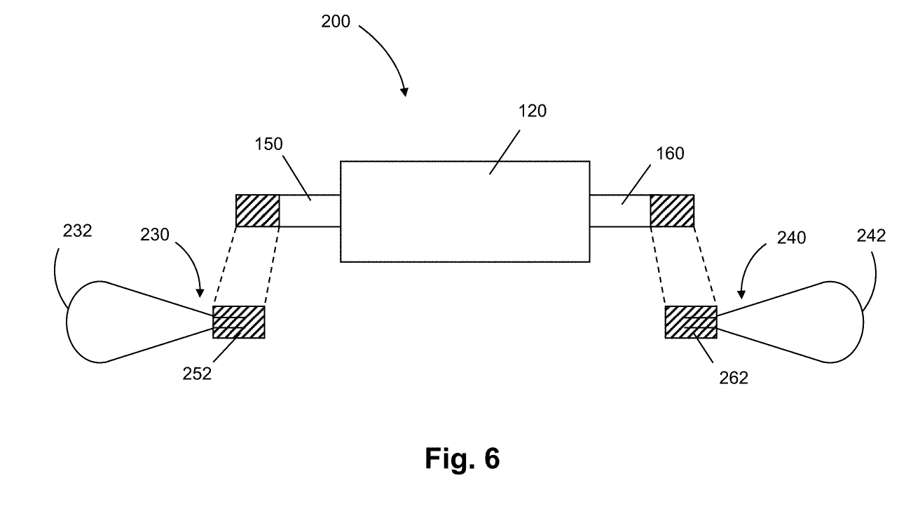

With reference to FIG. 6, the mouthpiece module 200 is configured to attach around the ears of the user to mount the mouthpiece 120 in position as previously described. Straps 230 and 240 are preferably identical and respectively mount an elastic loop 232 and 242. Each loop is dimensioned and configured so that they loop around the ears of the user. The elastic loops 232 and 242 are preferably formed from thin elastic and attached to fastener portions 252 and 262. The mouthpiece 120 may be similar in form and function to that described for the mouthpiece of FIG. 5.

While preferred embodiments of the foregoing have been set for purposes of illustration, the foregoing description should not be deemed a limitation of the invention herein. Accordingly, various modifications, adaptations and alternatives may occur to one skilled in the art without departing from the spirit and the scope of the present invention.

1. A mouthpiece module for use in conjunction with a nasal mask for a CPAP machine to seal the lips of a user comprising:
a mouthpiece having generally opposed first and second edges and opposed ends, said mouthpiece being formed of an absorbent flexible material wherein said ends are longitudinally spaced a distance greater than a lip interface when the lips of a user are in a closed position;
a pair of straps which extend from each of said opposed ends a distance sufficient to secure the straps to the back of the head of the user,
wherein when said straps are securable to the back of the head of the user, the mouthpiece extends along the interface of the lips and first and second portions of the module are wrappable around portions of each of the lips to provide an airtight seal at the mouth.

2. The mouthpiece module of claim 1 wherein said mouthpiece is formed of a cotton cloth material.

3. The mouthpiece module of claim 1 wherein said straps integrally extend from said opposed ends and are formed from substantially the same material as the mouthpiece.

4. The mouthpiece module of claim 1 wherein said mouthpiece is manufactured of material selected from the group consisting of cloth, polyester, hemp, cotton and a textile.

5. A method for sealing the air passageway of a mouth comprising:
providing a mouthpiece dimensioned to extend along a closed lip interface of the mouth and comprised of flexible absorbent material which is wrappable of the upper and lower lips; and
securing said mouthpiece to the head of the user by securing means connecting end portions of the mouthpiece and extending around the back of the head to provide an air seal at the mouth of the user.

6. The method of claim 5 further comprising:
disconnecting the mouthpiece from said securing means;
replacing the mouthpiece with a second mouthpiece; and
connecting securing means to said second mouthpiece.

7. A mouthpiece module for use in conjunction with a nasal mask for a CPAP machine to seal the lips of a user comprising:
a mouthpiece having generally opposed first and second edges and opposed ends, said mouthpiece being formed of an absorbent flexible material wherein said ends are longitudinally spaced a distance greater than a lip interface when the lips of a user are in a closed position;
a connector extending from each said end;
a pair of strap segments connectable to each connector and extending a distance sufficient to secure the straps to the user; and
a hook-and-loop fastener which connects each connector to a strap segment,
wherein when said strap segments are securable to the back of the head of the user, the mouthpiece extends along the interface of the lips and first and second portions of the mouthpiece are wrappable around portions of each of the lips to provide an airtight seal at the mouth.

8. The mouthpiece module of claim 7 wherein said connectors integrally extend from said opposed ends and are formed from substantially the same material as the mouthpiece.

9. The mouthpiece module of claim 7 wherein said mouthpiece is manufactured of material selected from the group consisting of cloth, polyester, hemp, cotton and a textile.

10. The mouthpiece module of claim 7 wherein a portion of each said hook-and-loop fabric fastener is attached to a portion of one of said strap segments.

11. The mouthpiece module of claim 7 wherein each said strap segment comprises a loop of elastic material.

12. The mouthpiece module of claim 1 wherein each said hook-and-loop fabric fastener is located adjacent to an end of said mouthpiece.

13. The mouthpiece module of claim 1 wherein said hook-and-loop fabric fastener is attached to a portion of both of said connectors adjacent to said opposed ends of the mouthpiece.

14. The mouthpiece module of claim 1 wherein said mouthpiece has a generally rectangular shape.

15. The mouthpiece module of claim 1 wherein said strap segments have a generally equal length and said connectors have a generally equal length.

16. A mouthpiece module for use in conjunction with a nasal mask for a CPAP machine to seal the lips of a user comprising:
a mouthpiece having generally opposed first and second edges and opposed ends, said mouthpiece being formed of an absorbent flexible material wherein said ends are longitudinally spaced a distance greater than a lip interface when the lips of a user are in a closed position;
securement means for securing said mouthpiece to the head of the user,
wherein when said securement means is securable to the head of the user, the mouthpiece extends along the interface of the lips and first and second portions of the mouthpiece are wrappable around portions of each of the lips to provide an airtight seal at the mouth.

17. The mouthpiece module of claim 1 wherein said mouthpiece is formed of a cotton cloth material.

18. The mouthpiece module of claim 1 wherein said securement means integrally extends from said opposed ends and are formed from substantially the same material as the mouthpiece.

19. The mouthpiece module of claim 1 wherein said mouthpiece is connectable to said securement means by a pair of hook-and-loop fabric connectors.

20. The mouthpiece module of claim 16 wherein said mouthpiece may be selectively disconnected from said securement means.

21. The mouthpiece module of claim 16 wherein said securement means comprises a band of elastic material.
